(19) United States
(12) Patent Application Publication
(10) Pub. No.: US 2004/0191196 A1
(43) Pub. Date: Sep. 30, 2004

(54) Novel conjugate compounds and dermatological compositions thereof

(76) Inventor: Dov Tamarkin, Maccabim (IL)

Correspondence Address: WILMER CUTLER PICKERING HALE AND DORR LLP, 60 STATE STREET, BOSTON, MA, 02109, US

(21) Appl. No.: 10/737,181

(22) Filed: Dec. 16, 2003

Related U.S. Application Data
(60) Provisional application No. 60/433,829, filed on Dec. 16, 2002.

(51) Int. Cl. A61K 7/135; A61K 31/59; A61K 31/355

(52) U.S. Cl. 424/62; 514/167; 514/179; 514/458

(57) ABSTRACT

The invention relates to novel cosmetic and dermatological compositions, comprising conjugate compounds, including a dicarboxylic acid moiety, which is covalently linked through covalent bonds to a biologically active alcohol, selected from the group of steroidal hormones, steroidal anti-inflammatory agents, vitamin E and vitamin D.

### RELATED APPLICATIONS

This application claims priority under 35 U.S.C. § 119(e) to co-pending application United States Patent and Trademark Application Ser. No. 60/433,829 filed on Dec. 16, 2002, and entitled “Novel Conjugate Compounds and Dermatological Compositions Thereof.”

### FIELD OF THE INVENTION

The invention relates to novel cosmetic and dermatological compositions, comprising conjugate compounds, including a dicarboxylic acid moiety, which is covalently linked through covalent bonds to a biologically active alcohol, selected from the group of steroidal hormones, steroidal anti-inflammatory agents, vitamin E and vitamin D.

### BACKGROUND OF THE INVENTION

Skin is a complex entity consisting of a variety of cells and organelles, each of which has a particular function. The pathways by which the cosmetic actives are absorbed and the role of the vehicle on the skin can be better understood if one is familiar with skin structure and function. Skin protects the body's organs from external environmental threats, including ultraviolet rays, and acts as a thermostat to maintain body temperature. It consists of several different layers, each with specialized functions. The major layers include the epidermis, the dermis and hypodermis. The epidermis is a stratifying layer of epithelial cells that overlies the dermis consisting of connective tissue layer. An internal layer of adipose tissue, the hypodermis, further supports the epidermis and dermis. Various factors influence the absorption of substances through the skin.

Cosmetic and pharmaceutical substances traverse the skin primarily either through the pores of the hair follicles, the sweat gland ducts or by passing through the protein/lipid domains of the stratum corneum. From the skin surface, the subsequent diffusion into the intra-cellular spaces and the cell takes place. In the initial transient diffusion stage, penetration occurs through the skin appendages, i.e. the hair follicles and the ducts. It then passes into the skin. The stratum corneum (SC) is a bio-membrane and distinguishes itself from the other membranes in the body in function and composition. It is made up of a matrix of protein-laden material surrounded by extracellular, multilamellar bilayers of lipid. The SC is less permeable for the lipophilic compounds compared to the water-soluble compounds. However, water-soluble molecules with low lipid solubility are usually thought to pass through the pores, whereas lipid-soluble materials pass through protein/lipid domains of the stratum corneum.

Hence, the route of penetration of a compound into and through the skin is determined, to a large extent, by the chemical structure of such compound. The present invention relates to compositions for topical cosmetic and dermatological treatment, comprising novel conjugates of a dicarboxylic acid and a biologically active alcohol, selected from the group of steroidal hormones, steroidal anti-inflammatory agents, vitamin E and vitamin D.

Dicarboxylic acids are known to possess a wide spectrum of biological activities, which can be beneficial in skin therapy, including the following. Such activities may be valuable in the treatment of a variety of skin disorders of both cosmetic and dermatological nature.

Dicarboxylic acids, having about 6 to 14 carbon atoms in their carbon atom skeleton have a variety of biologically relevant properties, useful in the treatment of skin disorders, as detailed below.

These compounds have demonstrated competitive inhibition of mitochondrial oxidoreductases and 5-α-reductase, which is responsible for the conversion of testosterone into dihydrotestosterone (DHT). DHT is involved in the control of various skin processes, such as keratinization, sebum production and hair growth patterns (S. Passi, M. Picardo, C. De Luca and M. Nazzaro-Porro, G. Ital. Dermatol. Venereol., 124 (1989) 455-63; D. Stamatiadis, M. C. Bulteau-Porotois and I. Mowszowicz, Inhibition of 5-α-reductase in human skin by zinc and azelaic acid, Br. J. Dermatol., 119 (1988) 627-632; S. Passi, M. Picardo, C. De Luca and M. Nazzaro-Porro, Mechanism of action of azelaic acid in the treatment of acne. G. Ital. Dermatol. Venereol. 124 (1989) 455-63).

These compounds have shown evidence of the inhibitory effect on the proliferation and cell viability of melanocytes and human melanoma cells (Breathnach A S, Robins E J, Nazzaro-Porro M, Passi S, Picardo M., Hyperpigmentary disorders—mechanisms of action. Effect of azelaic acid on melanoma and other tumoral cells in cultureActa Derm Venereol Suppl (Stockh) 1989; 143:62-6)

These compounds have also demonstrated a skin whitening effect (Rigoni C, Toffolo P, Serri R, Caputo R, Use of a cream based on 20% azelaic acid in the treatment of melasma, G Ital Dermatol Venereol 1989 Jan-Feb;124(1-2):I-VI); bacteriostatic activity to both aerobic and anaerobic bacteria including Propionibacterium acnes, anti-keratinizing activity, displaying antiproliferative cytostatic effects on keratinocytes and modulating the early and terminal phases of epidermal differentiation (M. Detmar, A. Mayer-da-Silva, R. Stadler and C. E. Orfanos, J Invest Dermatol 93 (1989) 70-4), regulation of the ADF/TRX gene expression (Y. U-Taniguchi, K. Furuke, H. Masutani, H. Nakamura and J. Yodoi, Oncol. Res. 7 (1995) 183-9), competitive inhibition of thioredoxin reductase (C. Kroll, A. Langner and H. H. Borchert, Free Radic. Biol. Med. 26 (1999) 850-7), and a sensitizing effect in the chemotherapeutic treatment of several melanoma cell lines. (J. Rodriguez-Vicente, V. Vicente-Ortega, and Canteras-Jordana, Pigment Cell Research, Volume 9, Number 6 December 1996).

Thus, azelaic acid has the potential to serve as a useful active agent for the treatment of various dermatological and cosmetic disorders, which involve inflammation, bacterial and fungal infection, pigmentation, cell hyperproliferation and different skin ageing phenomena.

It is generally accepted that oil-water partitioning characteristics of a chemical are crucial to its ability to penetrate the skin (R. H. Guy and J. Hadgraft, Pharm. Res. 5 (1988) 753). Skin penetration, and particularly penetration into the pilosebaceous unit, is directly correlated with oil solubility (lipophilicity). Consequently, a dicarboxylic acid, which is relatively hydrophilic, can barely penetrate the skin and thus, its therapeutic benefits are limited. This can theoretically be addressed by producing simple ester or amide derivatives of said dicarboxylic acid, wherein the counter moiety is a short to medium-chain alcohol or amine. Yet, despite many trials to employ simple esters of such dicarboxylic acids, none has been shown to be therapeutically superior to the naive dicarboxylic acid.

The same limitation prevails for many active cosmetic and pharmaceutical compounds, which have hydroxy or amine groups on their backbone, thus being at least partially hydrophilic.

Alpha-omega-dicarboxylic acids, and certain mercapto, ester and salt derivatives have been used in the treatment of a variety of skin disorders and/or conditions. Relevant discussions on their uses may be found in the following references.

Hill et al in U.S. Pat. No. 4,034,077 teaches the use of a composition comprising sebacic acid for the treatment of skin irritation and the prevention of diaper rash in which the dicarboxylic acid acts as a barrier between the urine and the skin and also neutralizes ammonia. It does not teach the use of sebacic acid in the treatment of any endogenous disorder, including any form of ichthyosis, or any hormonal imbalance.

Nazzaro-Porro (U.S. Pat. No. 4,292,326) discloses a method of treating hyperpigmentary dermatoses with dicarboxylic acids, such as azelaic acid. These acids, along with their mono- and dimercapto derivatives, are used for their ability to normalize skin color by inhibiting melanogenesis. Nazzaro-Porro (U.S. Pat. No. 4,386,104) teaches the use of the same compounds for the treatment of acne. It also teaches adding a small amount of keratolytic agent to the composition. Nazzaro-Porro (U.S. Pat. No. 5,385,943) also discloses the use of topically applied preparations, comprising an ester of a dicarboxylic acid cleavable by skin enzymes, particularly a glycerol ester, for treatment of presbyderma of the aging skin.

Thornfeldt (U.S. Pat. No. 4,885,282) discloses a treatment of hyperhydrosis, ichthyosis and wrinkling of the skin by means of a mono- or di-carboxylic acids (4-18C), along with their mercapto derivatives, salts and esters. The use of alkyl, polyol, oligosaccharide and polysaccharide esters, and specifically glycerol, polyethylene glycol, polypropylene glycol and sucrose esters of the respective mono- or di-carboxylic acids is described. UK Pat. Appl. No. GB 2,285,805 teaches the use of esters of dicarboxylic acids with vitamins A, E and D as antitumor agents. Chamness (U.S. Pat. No. 5,547,989) teaches a topical composition comprising dicarboxylic acids (7-13C and specifically AZA), salts and esters thereof for treating corns and calluses. However, no specific ester is claimed or demonstrated by an example.

Sugibayashi et al (Chem. Pharm. Bull. 36(4): 1519-1528,1988) teaches the use of penetration enhancers for the model compound indomethacin. It discloses the use of salicylates as enhancers because of their ability to soften and dissolve the stratum corneum. They teach the use of salicylates as keratolytic agents to remove the outer layer of cells, which then allows easier penetration of the desired compound.

Luedders, (U.S. Pat. No. 4,299,826) teaches a physical mixture of the antibacterial agent erythromycin, with the penetration enhancer diisopropyl sebacate. Luedders teaches that this additive increases the penetration of erythromycin. DE 4213419 discloses a salicylic acid ester derivative of azelaic acid in a glycerin trinitrate carrier for pharmaceutical applications. The low pH of the salicylic acid ester tends to irritate the treated tissues.

Known references disclose complex esters of straight chain dicarboxylic acids. In U.S. Pat. No. 5,494,924, Cavazza et al. teaches the treatment of ichthyoses using complex esters of .alpha.,.omega.-dicarboxylic acids and camitine. Bilibin et al. (USSR Pat. No. 761,452) teaches the synthesis of dicarboxylic acids esterified by reaction with p-hydroxy benzoates, which are used as monomers for the formation of liquid crystalline polymers. In U.S. Pat. No. 3,660,467, Gould teaches phenoxy phenyl esters of .alpha.,.omega.-dicarboxylic acids for use as synthetic lubricants and heat transfer fluids. Portnoy et al (Chemical Engineering Data Series, 1958, 3:287-293) teaches the use of phenyl dicarboxylates in the development of nonspreading lubricant oils. Although the aforementioned an describes compounds comprising esterified dicarboxylic acids, there is no discussion of the use of these compounds in treatment of skin disorders.

Tamarkin (U.S. Pat No. 6,180,669) teaches a compound effective for the treatment of dermatological disorders comprising a mono- or diester of an alpha, omega dicarboxylic acid, wherein the alcohol moiety of the said ester comprises a keratolytically active alcohol. According to U.S. Pat No. 6,180,669, a “keratolytically active alcohol moiety” means a compound which loosens and removes the stratum corneum of the skin or having an antikeratinizing effect via modulation of keratinocyte differentiation and growth, wherein the keratolytic agent moieties include phenol and substituted phenolic compounds, alpha-hydroxy acids and derivatives thereof, hydroxybenzoic acid and their ester, anhydride and amine derivatives, alkylhydroxybenzoate, dihydroxy benzene and their ester, anhydride and amide derivatives, cresols and their ester, anhydride and amide derivatives. Keratolytic agents According to U.S. Pat No. 6,180,669, also include alcohol derivatives of Vitamin A (retinoic acid), e.g., retinol and derivatives thereof.

Thus, there remains a need to provide therapeutic agents for the treatment of skin disorders, which demonstrate improved efficacy and reduced irritation over the agents available in the prior art.

### SUMMARY OF THE INVENTION

The present invention provides novel conjugate compounds effective in the treatment of skin disorders of cosmetic and dermatological nature. Such compounds can either be incorporated into pharmaceutical products, to be prescribed by medical professionals or in over the counter (OTC) or cosmetic preparations offered directly to customers for self-usage.

According to the invention, the conjugate compounds include an alpha, omega dicarboxylic acid moiety, which is covalently linked through ester bonds to a biologically active alcohol, selected from the group of steroidal hormones, steroidal anti-inflammatory agents, vitamin E and vitamin D, possessing a plurality of activities, which are unrelated to keratinization. The conjugate compound contain one dicarboxylic acid and one or two biologically active alcohol to provide either the respective mono- or di-conjugate. As such, the conjugate compound comprises two moieties, each capable of treating the symptoms of a variety of skin disorders or to improve the appearance of the skin. The conjugate compound possesses the additional advantage of providing the two moieties in a form, which penetrates more rapidly into a dermal site.

### BRIEF DESCRIPTION OF THE DRAWING

The invention is described with reference to the following drawings which are presented for the purpose of illustration only and which are not intended to be limiting of the invention.

FIG. 1 of a hydroxyl corticosteroid molecule indicating carbon naming convention.

FIG. 2 is an illustration of covalent linkage sites between a steroid hormones and the dicarboxylic acid to yield a desirable conjugate compound according to one or more embodiments of the present invention.

FIG. 3 is an exemplary illustration of covalent linkage sites between a corticosteroid and the dicarboxylic acid, to yield a desirable conjugate compound, according to one or more embodiments of the present invention.

FIG. 4 is a schematic illustration of a dicarboxylic acid—steroid conjugate according to one or more embodiments of the present invention.

FIG. 5 illustrates exemplary tocopherol isomers and tocotrienol isomers for use in one or more embodiments of the present invention.

FIG. 6 is a schematic illustration of exemplary conjugates of a dicarboxylic acid and vitamin E according to one or more embodiments of the present invention.

FIG. 7 is a schematic illustration of exemplary dicarboxylic acid—Vitamin D conjugates according to one or more embodiments of the present invention.

### DETAILED DESCRIPTION OF THE INVENTION

The terms “dicarboxylic acid”, “alpha, omega dicarboxylic acid” and “dicarboxylic acid moiety” are used herein synonymously, to mean a straight carbon chain terminating on both ends with a carboxylic acid functional group. The length of the dicarboxylic acid moiety of the conjugate is about 6 to 14 carbon atoms. In a preferred embodiment, the dicarboxylic acid moiety comprises between 8 and 10 carbons. The carbon chain backbone may be saturated or unsaturated. In preferred embodiments, the unsaturated backbone may contain 1-3 double bonds. The straight carbon chain also may be substituted, for example, it may be linked to hydrocarbon groups along the carbon atom backbone. Suitable dicarboxylic acid moieties include, but are not limited to, adipic acid, pimelic acid, suberic acid, azelaic acid, sebacic acid, 1,11-undecanedioic acid, 1,12-dodecanedioic acid, 1,13-tridecanedioic acid and 1,14-tetradecanedioic acid. In a preferred embodiment, the dicarboxylic acid is azelaic acid. Suitable substitutions along the carbon chain backbone include, but are not limited to, alkyl, aryl, alkenyl, and benzyl groups. By way of example only, suitable hydrocabons, e.g., aryl and alkyl substituents, include methyl, ethyl, propyl, phenyl, benzyl and the like.

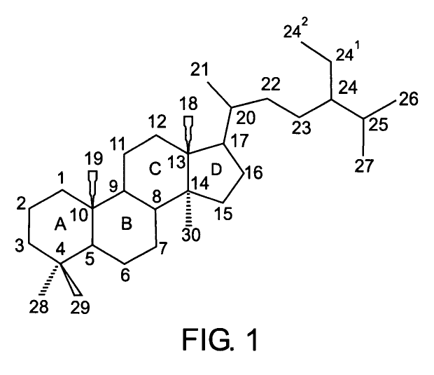

The terms “steroid hormones”, “hydroxy steroidal anti-inflammatory agents” and “hydroxy corticosteroids (hereinafter Hydroxy CS)” are used herein collectively to include all naturally occurring and steroid medications, having at least one hydroxyl group on their carbon skeleton. Basic structures of CS consist of the 21 carbon-atom ring structure of sterols (FIG. 1). The activity of CS is dramatically enhanced by the introduction of an unsaturated bond between the first two carbon atoms, by the nature of the side chains, particularly on the 21 C position, and by halogenation of the 9-alpha position.

Steroids are carried through the bloodstream bound to a protein to keep them soluble. Since they are lipophilic, they can traverse cell membranes. Once inside a cell they may bind to a receptor and the complex may then interact with DNA to modulate transcription, turning genes off or on. Some steroids work in a different fashion by binding to ion channels and changing the cell's permeability to specific ions, which may in turn have a variety of effects on the cell.

Estrogens (e.g., estradiol, estrone, estriol), Androgens (e.g., testosterone, DHEA, androstenedione) and Progesterone, and their artificial, naturally occurring or synthetic analogs play an important role in tissue regeneration, especially the skin, bones, and muscles. They are involved in maintenance of lean body mass, bone density, skin elasticity, sex drive and cardiovascular health in both sexes.

Corticosteroids (CS) are commonly used for treatment of various inflammatory skin conditions. CS reduce or even completely suppress the symptoms of inflammation and alleviate accompanying symptoms such as pain, itching and paresthesia. CS inhibit the release of phospholipase 1, the enzyme responsible for liberation of arachidonic acid from phospholipids which are constituents of the cell membranes. As a consequence formation of prostaglandin's (PG) and other derivatives of arachidonic pathway is inhibited. PG derive from arachidonic acid via the cyclooxigenase pathway, they contribute to the inflammation of the skin in contact allergic eczema, psoriasis and UV induced inflammation. They also enhance the itch induced by histamine. In addition the CS exhibit also antiproliferative and immunosuppressive effects. In view of such broad therapeutic effects a field of indications is open to the application of CS in dermatology:

Table 1 lays out, in a non-limiting fashion, examples of Steroid Hormones and hydroxy corticosteroids, which can be linked to a dicarboxylic acid according to the present invention

TABLE 1
Examples of Steroid Hormones and hydroxy corticosteroids,
which can be linked to a dicarboxylic acid
Steroid Hormones with One Hydroxy Group

| | |
|---|---|
| Cholesterol | 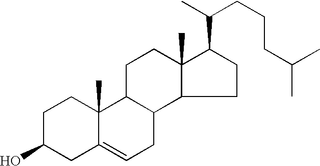 |
| 7-Dehydro- cholesterol (Provitamin D) | 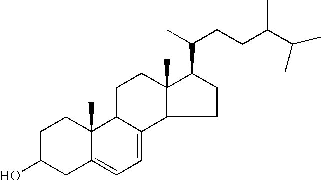 |
| Ergosterol | 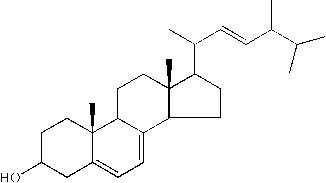 |
| Pregnonolone | 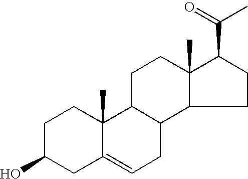 |
| Dehydro- epiandrosterone | 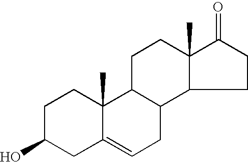 |
| Testosterone | 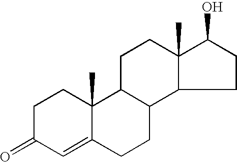 |
| Estrone | 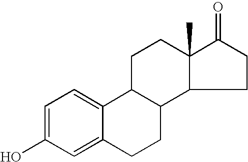 |

Steroid Hormones with Two Hydroxy Groups

| | |
|---|---|
| Cortisone | 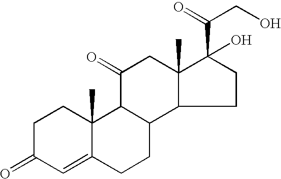 |
| Corticosterone | 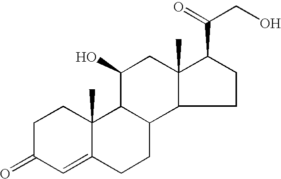 |
| 17 beta Estradiol | 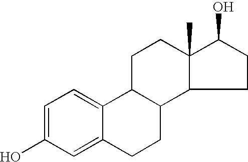 |
| Aldosterone | 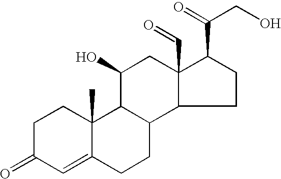 |

Steroid Hormones with Three Hydroxy Groups

| | |
|---|---|
| Cortisol | 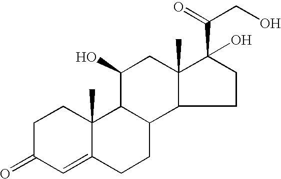 |
| Estriol | 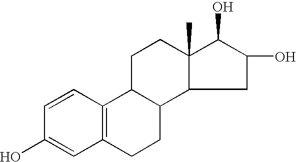 |

Synthetic Corticosteroids

| | |
|---|---|
| Hydrocortisone | 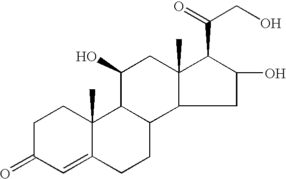 |
| 9-alpha-Fluoro- cortisole | 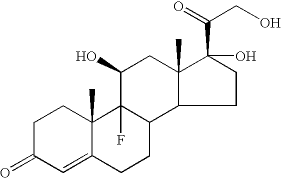 |
| Prednisolone | 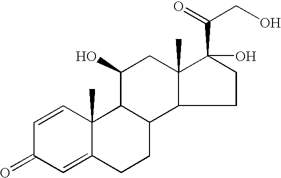 |
| Prednisone | 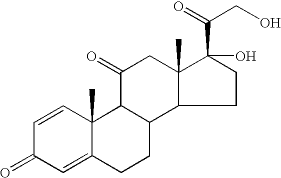 |
| Betamethasone | 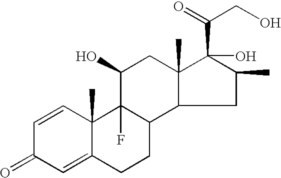 |
| Dexamethasone | 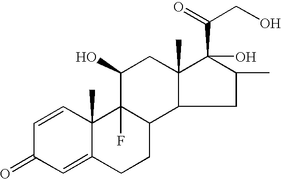 |
| Triamcolone | 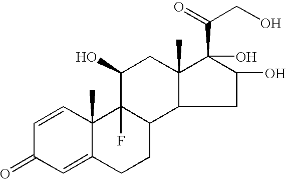 |

Conjugation of one or two such hormones molecules with a dicarboxylic acid through ester bonds forms a conjugate compound with modified skin penetration and bioavailability properties.

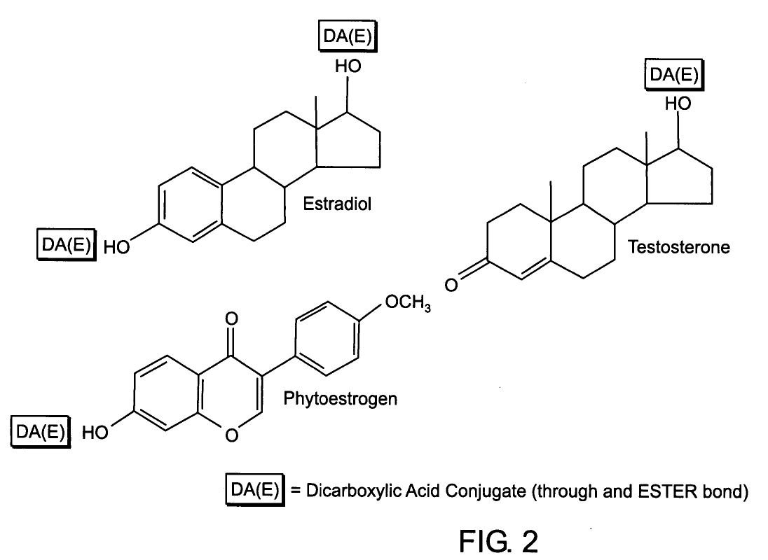

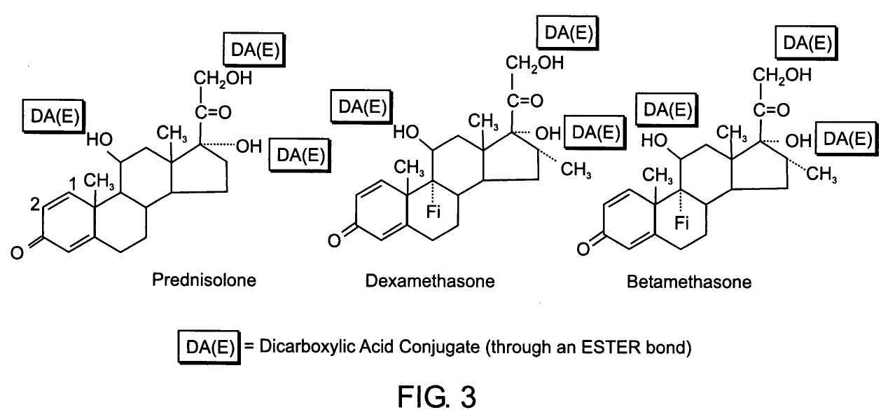

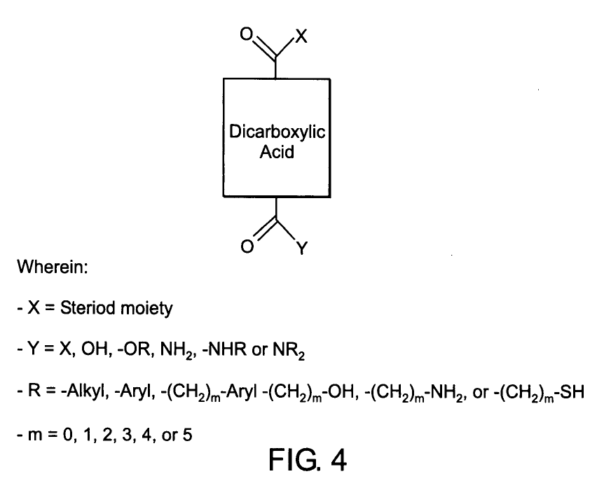

FIGS. 2 and 3 displays, in an exemplary fashion, a series of steroid hormones and annotates the possible covalent linkage sites between such steroids and the dicarboxylic acid, to yield a desirable conjugate compound. The dicarboxylic acid can be linked covalently through an ester bond with one or two steroid hormone or CS molecules, as demonstrated in FIG. 4. For one ester linkage, X=steroid hormone or CS; and for two linkages, X=Y=steroid hormone or CS. In case of a single steroid linkage, the remaining carboxylic group of the dicarboxylic acid can remain in its acidic state (Y=—OH) or be derivatized, wherein Y is selected from the group of, —OR, —NH₂, —NHR or NR₂; R can be selected from the group of -Alkyl, -Aryl, —(CH₂)ₘ—Aryl —(CH₂)ₘ—OH, —(CH₂)ₘ—NH₂, or —(CH₂)ₘ—SH; and m can be 0,1, 2, 3, 4 or 5.

The dicarboxylic acid—steroid or CS conjugate of the present invention is useful in the treatment of a variety of dermatological disorders, due to the simultaneous presence of the two active moieties, i.e., the dicarboxylic acid and the steroid in the same molecule. Upon skin penetration, the conjugate molecule can exert a joined, and conceivable a synergistic effect of the two moieties, either via enzymatic or chemical hydrolysis, thereby releasing the two moieties in the same skin site, or through simultaneous effects of the two moieties, while linked in very close proximity to each other. Each of the two moieties of the conjugate contributes to the therapeutic affect, via a different mechanism: The dicarboxylic acid exerts its inhibitory effect on immune cell migration, while the CS contributes an anti-inflammatory effect through its inhibition of the prostaglandin synthesis cascade.

Psoriasis is a very common chronic skin disease, which may be the target of treatment using the dicarboxylic acid—steroid conjugate of the present invention. It is marked by periodic flare-ups of sharply defined red patches covered by a silvery, flaky surface. Examples of other inflammatory disorders, which can be treated by the dicarboxylic acid—steroid conjugate of the present invention are atopic dermatitis, seborrhea, seborrheic dermatitis of the face and trunk, seborrheic blepharitis, contact dermatitis, stasis dermatitis (gravitational eczema; varicose eczema), exfoliative dermatitis (erythroderma), lichen simplex chronicus, pityriasis rosea and pemphigus.

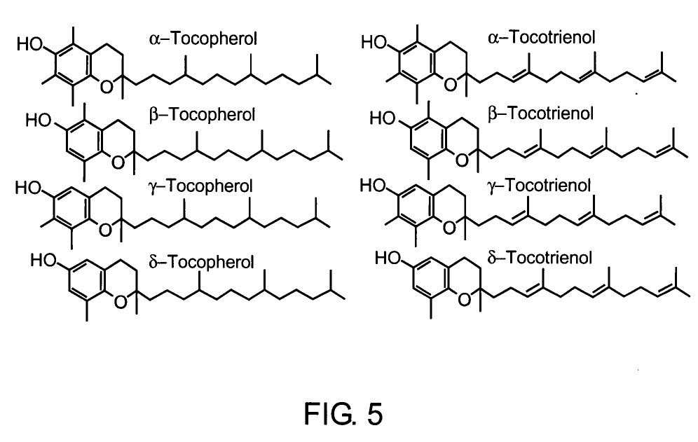

The term “Vitamin E” refers to a class of lipid-soluble antioxidants consisting of four tocopherol isomers and four tocotrienol isomers, i.e., alpha-, beta-, delta- and gamma-tocopherol and alpha-, beta-, delta- and gamma-tocotrienol (FIG. 5).

Vitamin E is present in all cell membranes, plasma lipoproteins and red blood cells. As the major lipid-soluble chain-breaking antioxidant in humans, it functions to protect DNA, low-density lipoproteins (LDL) and polyunsaturated fatty acids (PUFAs) from free radical-induced oxidation. Vitamin E also quenches singlet oxygen. In this regard, d-alpha-tocopherol is the most biologically active isomer.

This antioxidant capability is then also great in helping to prevent degenerative diseases—including heart disease, strokes, arthritis, senility, diabetes and cancer. It also assists in fighting heart disease and cancers and is essential for red blood cells, helps with cellular respiration and protects the body from pollution—especially the lungs. Vitamin E is also useful in preventing blood clots from forming and promotes fertility, reduces and/or prevents hot flushes in menopause. An increase in stamina and endurance is also attributed to Vitamin E.

Vitamin E is also used topically to great effect for skin treatments—in helping the skin look younger, promoting healing and cutting down the risk of scar tissue forming. Used on the skin it is also reported to help with eczema, skin ulcers cold sores and shingles.

While being one of the most effective forms of vitamin E, it is also quite unstable, and thus, conjugating it with a organic acids, such as acetic acid, to product tocopherol acetated α-(ATA) and succinic acid (to product tocopherol succinate) have been used. Such esters can be hydrolyzed to the free active form by skin-related esterases, as demonstrated in several literature reports.

Linkage of a dicarboxylic acid and vitamin E together is due to result in higher skin bioavailability of the two moieties. Upon skin penetration, the conjugate molecule can exert a joined, and conceivable a synergistic affect of the two moieties, either via enzymatic or chemical hydrolysis, thereby releasing the two moieties in the same skin site, or through simultaneous effects of the two moieties, while linked in very close proximity to each other.

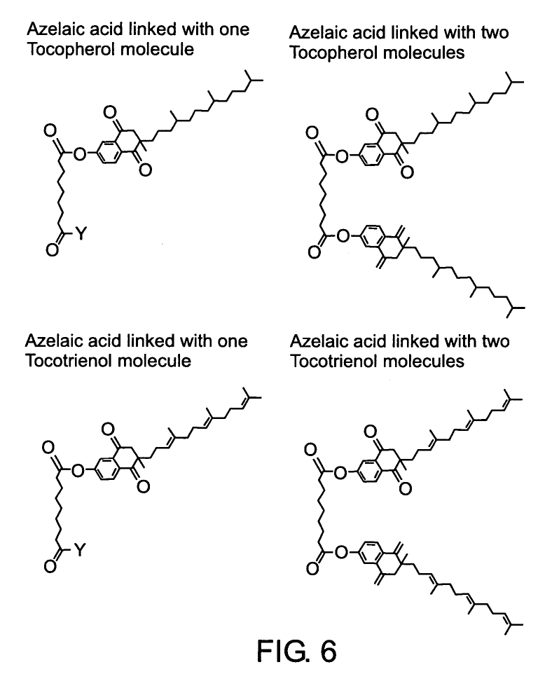

The dicarboxylic acid can be linked with one or two vitamin E molecules, as Exemplified in FIG. 6, wherein the dicarboxylic acid is azelaic acid. In case of a single vitamin D linkage, the remaining carboxylic group of the dicarboxylic acid can remain in its acidic state (Y=—OH) or be derivatized, wherein Y is selected from the group of, —OR, —NH₂, —NHR or NR₂; R can be selected from the group of -Alkyl, -Aryl, —(CH₂)ₘ-Aryl —(CH₂)ₘ—OH, —(CH₂)ₘ—NH₂, or —(CH₂)ₘ—SH; and m can be 0,1, 2, 3, 4 or 5.

Provitamin D is a simple derivative of cholesterol, which occurs when a hydrogen is removed from the number 7 carbon (see FIG. 1), which then forms a double bond with the number 8 carbon, in the second, or ‘B’ ring of the cholesterol molecule. The cholesterol is ‘oxidized’ (that is, an electron is removed with the hydrogen atom), so that the double bond is a consequence of 2 mutually shared electrons between carbons 7 and 8. It is converted to Vitamin D₃ by the action of ultraviolet light through our skin. In this reaction, the B ring of the sterol molecule is opened.

Vitamin D analogues have a range of dermatological effects that, via specific binding to the vitamin D receptors and also non-receptor-mediated events, are relevant to their therapeutic efficacy in diseases such as psoriasis. These effects include inhibition of keratinocyte proliferation; enhancement of normal keratinization; and inhibition of accumulation of inflammatory cells, particularly neutrophils and T lymphocytes.

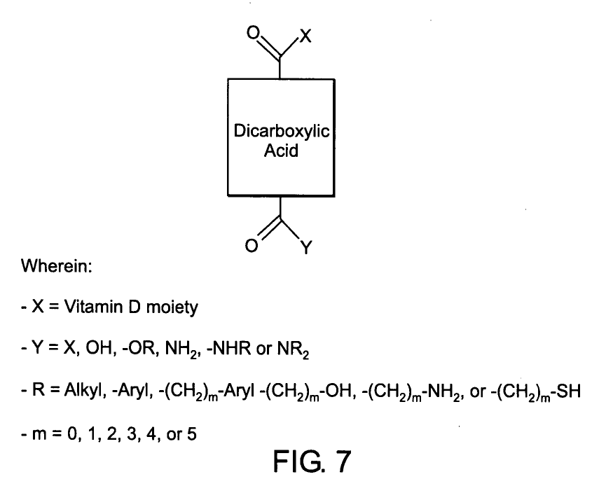

Table 2 lays out, in a non-limiting fashion, examples of vitamin D moieties, which can be linked to a dicarboxylic acid according to the present invention. The dicarboxylic acid can be linked covalently through an ester bond with one or two vitamin D moieties, as demonstrated in FIG. 7. For one ester linkage, X=vitamin D; and for two linkages, X=Y=vitamin D. In case of a single vitamin D linkage, the remaining carboxylic group of the dicarboxylic acid can remain in its acidic state (Y=—OH) or be derivatized, wherein Y is selected from the group of, —OR, —NH₂, —NHR or NR₂; R can be selected from the group of -Alkyl, -Aryl, —(CH₂)ₘ-Aryl —(CH₂)ₘ—OH, —(CH₂)ₘ—NH₂, or —(CH₂)ₘSH; and m can be 0,1, 2, 3, 4 or 5.

TABLE 2
Examples of Steroid Hormones and hydroxy corticosteroids,
which can be linked to a dicarboxylic acid

| | |
|---|---|
| Vitamin D2 - Calciferol | 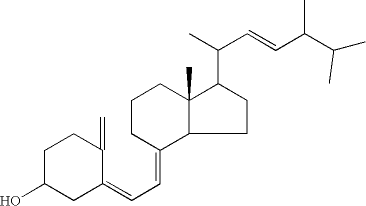 |
| Vitamin D3 - Cholecalciferol | 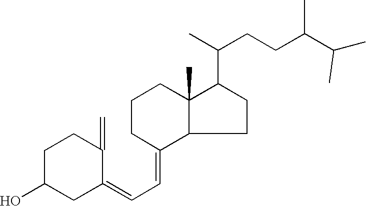 |
| 25 Hydroxycholecalciferol | 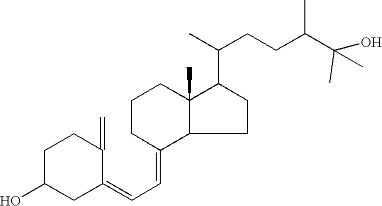 |
| 1-25 Dihydroxycholecalciferol | 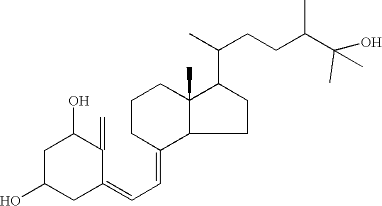 |

Compositions to include the conjugate compounds can be in many formulation forms including, but not limited to, liquids, solutions, lotions, creams, pastes, emulsions, gels, soap bars, foams, sprays or aerosols. The conjugate compound it to be incorporated in said formulation form in a concentration, which is sufficient to treat the designated cosmetic or dermatological skin disorder.

The conjugate compounds are useful in the treatment of skin disorders of cosmetic and dermatological nature.

Examples dermatological disorders of cosmetic or aesthetic nature are set forth in the following list: aging skin, dry skin, scaly skin, sun damaged skin, oily skin, fine lines and wrinkles, age spots, various hyperpigmented spots, melasma, puffy eyes, acne, redness of the skin, spider veins telangiectasia, atrophic and hypertropic scar, cellulite and obesity.

Because of the plurality of therapeutic affects, exerted by the conjugate of the present invention, it is particularly useful for the therapy (prevention, treatment, alleviating the symptoms of, or cure) of a wide variety of dermatological disorders (also termed “dermatoses” or “dermatosis”), especially, such disorders that involve a plurality of etiological factors.

By including an appropriate conjugate agent, in a therapeutically effective amount, in the formulation, the compositions of the present invention are useful in the therapy of a variety of dermatological disorders, which involve inflammation, bacterial and fungal infection, hyperpigmentation, hyperkeratinization, hypertrophy of the stratum corneum, excess sebum secretion, microbial infection, dermatophytoses, or increased conversion of testosterone to dihydrotestosterone. The conjugate can be used as mono-therapy of said disorder, or in combination with other drugs, as required for the prevention, treatment, alleviating the symptoms of, or cure of said disorder. Such disorders are classified, in a non-limiting exemplary manner, according to the following groups:

Dermatitis such as contact dermatitis, atopic dermatitis, seborrheic dermatitis, nummular dermatitis, chronic dermatitis of the hands and feet, generalized exfoliative dermatitis, stasis dermatitis and lichen simplex chronicus.

Bacterial infections such as cellulitis, impetigo, acute lymphangitis, lymphadenitis, erysipelas, cutaneous abscesses. necrotizing subcutaneous infections, staphylococcal scalded skin syndrome, folliculitis, furuncles, hidradenitis suppurativa, carbuncles, paronychial infections, and erythrasma.

Fungal Infections such as dermatophyte Infections, and Yeast Infections. parasitic infections such as scabies, pediculosis, and creeping eruption, and viral infections

Disorders of hair follicles and sebaceous glands such as acne, rosacea, perioral dermatitis, hypertrichosis (hirsutism), alopecia, pseudofolliculitis barbae, and keratinous cyst.

Scaling papular diseases such as psoriasis, pityriasis rosea, lichen planus, and pityriasis rubra pilaris.

Reactions to sunlight such as sunburn, chronic effects of sunlight, andphotosensitivity.

Pigmentation disorders such as hypopigmentation, , vitiligo, albinism, postinflammatory hypopigmentation, hyperpigmentation, melasma (chloasma), drug-induced hyperpigmentation, and postinflammatory hyperpigmentation.

Disorders of comification such as ichthyosis, keratosis pilaris, calluses and corns, and actinic keratosis.

Inflammatory reactions such as drug eruptions, toxic epidermal necrolysis, erythema multiforme, erythema nodosum, and granuloma annulare.

It would be apparent to those of ordinary skill in the art of cosmetics and dermatology that additives to such compositions may be selected from but are not limited to the group consisting of water, surfactants, emulsifiers, diglycerides, triglycerides, stabilizing agents, thickening agents, alpha-hydroxy carboxylic acids, antioxidants, preservatives, moisturizers, petroleum, mineral oil, glycerol, ethanol, propanol, isopropanol, butanol, polymeric gelling agents, flavoring, colorant and odorant agents and other formulation components, used in the art of pharmaceutical and cosmetic formulary. The following groups of additives can be incorporated in a composition of the present invention, in addition to the conjugate compounds:

1. Other cosmetic or pharmaceutical active agents.

2. Materials, which can improve the effect of the conjugates.

3. Release and controlled delivery materials, which can prolong the time of action, for example by way of microencapsulation, which results in slow release. Additives of this nature include various surfactants and phospholipids.

4. Formulation materials, which include solvents, surfactants, antimicrobial preservatives, antioxidants and fragrance materials.

The terms “therapy” and “treatment” as used herein interchangeably, cover any treatment of a disease or disorder, and includes, for example:

(i) Curing the disease or disorder;

(ii) preventing the disease or disorder from occurring in a subject which may be predisposed to the disease but has not yet been diagnosed as having it;

(iii) inhibiting the disease or disorder, i.e. arresting its development; and

(iv) relieving the disease or disorder, i.e. causing regression of the disease.

What is claimed is:

1. A method of treating dermatological disorders, comprising:
administering topically to a subject having said dermatological disorder a therapeutically effective amount of a compound comprising a mono- or diester of an alpha, omega dicarboxylic acid, wherein at least one ester moiety of the said compound comprises a active alcohol moiety, selected from the groups comprising steroid hormones, corticosteroids, vitamin E and vitamin D.

2. The method of claim 1, wherein said compound is applied topically to an affected area.

3. The method of claim 1, wherein said disorder involves inflammation, bacterial and fungal infection, hyperpigmentation, hyperkeratinization, hypertrophy of the stratum corneum, excess sebum secretion, microbial infection, dermatophytoses, or increased conversion of testosterone to dihydrotestosterone.

4. The method of claim 1, wherein said disorder involves aging skin, dry skin, scaly skin, sun damaged skin, oily skin, fine lines and wrinkles, age spots, various hyperpigmented spots, melasma, puffy eyes, acne, redness of the skin, spider veins telangiectasia, atrophic and hypertropic scar, cellulite and obesity.

5. The method of claim 1, wherein said disorder involves a plurality of etiological factors.

6. The method of claim 1, wherein said disorder is selected from the group comprising:
dermatitis, contact dermatitis, atopic dermatitis, seborrheic dermatitis, nummular dermatitis, chronic dermatitis of the hands and feet, generalized exfoliative dermatitis, stasis dermatitis, lichen simplex chronicus, bacterial infections, cellulites, acute lymphangitis, lymphadenitis, erysipelas, cutaneous abscesses, necrotizing subcutaneous infections, staphylococcal scalded skin syndrome, folliculitis, furuncles, hidradenitis sppurativa, carbuncles, paronychial infections, erythrasma, impetigo, fungal infections, dermatophyte infections, yeast infections, viral infections, acne, rosacea, perioral dermatitis, hypertrichosis, hirsutism, alopecia, psoriasis, pityriasis rosea, lichen planus, pityriasis rubra pilaris, sunburn, chronic effects of sunlight, photosensitivity, hypopigmentation, vitiligo, albinism, postinflammatory hypopigmentation, hyperpigmentation, melasma (chloasma), drug-induced hyperpigmentation, postinflammatory hyperpigmentation, disorders of cornification, ichthyosis, keratosis pilaris and eczema

7. The method of claim 1, wherein the alpha, omega dicarboxylic acid comprises 6 to 14, preferably 8 to 10, carbon atoms in its carbon chain backbone.

8. The method of claim 1, wherein said alpha, omega dicarboxylic acid comprises azelaic acid.

9. The method of claim 1, wherein the alpha, omega dicarboxylic acid carbon chain backbone is unsaturated.

10. The method of claim 1, wherein the alpha, omega dicarboxylic acid moiety is linked to a hydrocarbon substituent.

11. The method of claim 1, wherein the alpha, omega dicarboxylic acid moiety is substituted by alkyl, aryl, alkenyl or benzyl groups.

12. The method of claim 1, wherein said alpha, omega dicarboxylic acid is selected from the group consisting of adipic acid, pimelic acid, suberic acid, azelaic acid, sebacic acid, 1,11-undecanedioic acid, 1,12-dodecanedioic acid, 1,13-tridecanedioic acid and 1,14-tetradecanedioic acid.

13. A compound for the treatment of a skin disorder, comprising an alpha, omega dicarboxylic acid, covalently linked through an ester bond with at least one active alcohol moiety, selected from the group comprising steroid hormones, corticosteroids, vitamin E and vitamin D.

14. The compound of claim 13, wherein the alpha, omega dicarboxylic acid comprises 6 to 14, preferably 8 to 10, carbon atoms in its carbon chain backbone.

15. The compound of claim 13, wherein said alpha, omega dicarboxylic acid is selected from the group consisting of adipic acid, pimelic acid, suberic acid, azelaic acid, sebacic acid, 1,1 1-undecanedioic acid, 1,12-dodecanedioic acid, 1,13-tridecanedioic acid and 1,14-tetradecanedioic acid.

16. The compound of claim 13, wherein said alpha, omega dicarboxylic acid comprises azelaic acid.

17. The compound of claim 13, wherein the alpha, omega dicarboxylic acid carbon chain backbone is unsaturated.

18. The method of claim 17, wherein the backbone comprises about one to three double bonds.

19. The compound of claim 13, wherein the alpha, omega dicarboxylic acid moiety is linked to a hydrocarbon substituent.

20. The compound of claim 13, wherein the alpha, omega dicarboxylic acid moiety is substituted by alkyl, aryl, alkenyl or benzyl groups.

21. A pharmaceutical or cosmetic composition, comprising a therapeutically effective amount of a compound, comprising an alpha, omega dicarboxylic acid covalently linked through an ester bond with at least one active alcohol moiety, selected from the group comprising steroid hormones, corticosteroids, vitamin E and vitamin D and a pharmaceutically or cosmetically acceptable carrier.

22. The pharmaceutical or cosmetic composition of claim 21, wherein the alpha, omega dicarboxylic acid comprises 6 to 14, preferably 8 to 10, carbon atoms in its carbon chain backbone.

23. The pharmaceutical or cosmetic composition of claim 21, wherein said alpha, omega dicarboxylic acid is selected from the group consisting of adipic acid, pimelic acid, suberic acid, azelaic acid, sebacic acid, 1,1 1-undecanedioic acid, 1,12-dodecanedioic acid, 1,13-tridecanedioic acid and 1,14-tetradecanedioic acid.

24. The pharmaceutical or cosmetic composition of claim 21, wherein said alpha, omega dicarboxylic acid comprises azelaic acid.

25. The pharmaceutical or cosmetic composition of claim 21, wherein the alpha, omega dicarboxylic acid carbon chain backbone is unsaturated.

26. The pharmaceutical or cosmetic composition of claim 25, wherein the backbone comprises about one to three double bonds.

27. The pharmaceutical or cosmetic composition of claim 21, wherein the alpha, omega dicarboxylic acid moiety is linked to a hydrocarbon substituent.

28. The pharmaceutical or cosmetic composition of claim 21, wherein the alpha, omega dicarboxylic acid moiety is substituted by alkyl, aryl, alkenyl or benzyl groups.

29. The composition of claim 21, wherein said therapeutically effective amount of said compound comprises an amount effective to treat a dermatological or cosmetic disorder.

30. The composition of claim 29, wherein said disorder involves inflammation, bacterial and fungal infection, hyperpigmentation, hyperkeratinization, hypertrophy of the stratum corneum, excess sebum secretion, microbial infection, dermatophytoses, or increased conversion of testosterone to dihydrotestosterone.

31. The composition of claim 29, wherein said disorder is selected from the group, comprising aging skin, dry skin, scaly skin, sun damaged skin, oily skin, fine lines and wrinkles, age spots, various hyperpigmented spots, melasma, puffy eyes, acne, redness of the skin, spider veins telangiectasia, atrophic and hypertropic scar, cellulite and obesity.

32. The composition of claim 29, wherein said disorder is selected from the group comprising:
dermatitis, contact dermatitis, atopic dermatitis, seborrheic dermatitis, nummular dermatitis, chronic dermatitis of the hands and feet, generalized exfoliative dermatitis, stasis dermatitis, lichen simplex chronicus, bacterial infections, cellulites, acute lymphangitis, lymphadenitis, erysipelas, cutaneous abscesses, necrotizing subcutaneous infections, staphylococcal scalded skin syndrome, folliculitis, furuncles, hidradenitis sppurativa, carbuncles, paronychial infections, erythrasma, impetigo, fungal infections, dermatophyte infections, yeast infections, viral infections, acne, rosacea, perioral dermatitis, hypertrichosis, hirsutism, alopecia, psoriasis, pityriasis rosea, lichen planus, pityriasis rubra pilaris, sunburn, chronic effects of sunlight, photosensitivity, hypopigmentation, vitiligo, albinism, postinflammatory hypopigmentation, hyperpigmentation, melasma (chloasma), drug-induced hyperpigmentation, postinflammatory hyperpigmentation, disorders of cornification, ichthyosis, keratosis pilaris and eczema

33. The composition of claim 21, wherein said compound is functional to release a plurality of dermatologically-active compounds when delivered to a target site of the skin.

34. The composition of claim 21, wherein said composition is in a form selected from the group consisting of liquid, solution, lotion, cream, paste, emulsion, gel, soap bar, foam, spray and aerosol.
